(19) United States
(12) Patent Application Publication
(10) Pub. No.: US 2006/0093646 A1
(43) Pub. Date: May 4, 2006

(54) Orthopedic and dental implant devices providing controlled drug delivery

(76) Inventors: Michael J. Cima, Winchester, MA (US); John T. Santini JR., North Chelmsford, MA (US); Mark A. Staples, Cambridge, MA (US); Charles E. Hutchinson, Canaan, NH (US)

Correspondence Address: SUTHERLAND ASBILL & BRENNAN LLP, 999 PEACHTREE STREET, N.E., ATLANTA, GA, 30309, US

(21) Appl. No.: 11/262,413

(22) Filed: Oct. 28, 2005

Related U.S. Application Data
(60) Provisional application No. 60/623,079, filed on Oct. 28, 2004.
(60) Provisional application No. 60/668,517, filed on Apr. 5, 2005.
(60) Provisional application No. 60/670,487, filed on Apr. 12, 2005.

(51) Int. Cl. A61F 2/28; A61F 2/02

(52) U.S. Cl. 424/425; 623/23.51; 606/76; 424/426

(57) ABSTRACT

Implantable prosthetic devices are provided for controlled drug delivery, for orthopedic and dental applications. The device may include a prosthetic device body having at least one outer surface area; two or more discrete reservoirs located in spaced apart positions across at least a portion of the outer surface area, the reservoirs formed with an opening at the surface of the device body and extending into the device body; and a release system disposed in the reservoirs which comprises at least one therapeutic or prophylactic agent, wherein following implantation into a patient the therapeutic or prophylactic agent is released in a controlled manner from the reservoirs. The prosthetic device body preferably is a joint prosthesis or part thereof, such as a hip prosthesis, a knee prosthesis, a vertebral or spinal disc prosthesis, or part thereof. Optional reservoir caps may further control release kinetics.

### CROSS-REFERENCE TO RELATED APPLICATIONS

This application claims the benefit of U.S. Provisional Application No. 60/623,079, filed Oct. 28, 2004; U.S. Provisional Application No. 60/668,517, filed Apr. 5, 2005; and U.S. Provisional Application No. 60/670,487, filed Apr. 12, 2005. Those applications are incorporated herein by reference in their entirety.

### BACKGROUND OF THE INVENTION

This invention is generally in the field of implantable medical and dental devices for controlled release of therapeutic and prophylactic agents into a human or animal patient, and particularly implants for replacing or augmenting bone, cartilage, or dental tissues.

Approximately 180,000 hip replacements are performed each year in the United States. Artificial joints have become a common therapeutic option for replacing the structure of, and restoring function to, injured or diseased joints, including hips, knees, elbows, and shoulders. A few examples of these implantable prosthetic joint devices are described in U.S. Pat. No. 6,436,148,which discloses a joint prosthesis having an overall contour and surface geometry which optimize fixation properties, and U.S. Pat. No. 6,503,281, which discloses a prosthetic assembly for a total hip replacement. These patents are expressly incorporated herein by reference. Risks that may follow the replacement surgery include infection and, in the long term with some types of devices, loss of bone tissue at the interface with the prosthetic device as the bone remodels and consequent loosening of the joint/prosthetic. It would be desirable to deliver one or more drugs locally at the implant site over an extended period of time following implantation of the prosthesis. It would also be desirable to control tissue growth at or into the prosthesis.

Another drawback of joint replacement is that the prosthetic implant eventually will wear out, for example, ten to twenty years following implantation. This is problematic where the patient receiving the joint replacement is relatively young and might be expected to live well beyond the useful life of the joint prosthetic. Replacement of the prosthetic may not be possible in some instances, and would nevertheless require another invasive surgery. It therefore also is desirable to provide implant devices and methods for extending the useful life of a patient's natural bone, joint, or cartilage, so that the need for a complete tissue replacement (e.g., total knee or hip replacement) can be substantially delayed.

U.S. Pat. No. 6,736,849 discloses a spinal implant prosthetic device, which may be provided with a coating comprising an antibiotic or growth factor. Coatings, however, substantially limit the selection of the coating materials and the drugs, as well as substantially limiting the control over the release kinetics and spatial release patterns. It would be desirable to deliver one or more drugs locally at the implant site over an extended period of time following implantation of the prosthesis. It would also be desirable to improve the control over the release kinetics and to provide a means for providing more complex release profiles and patterns, both temporally and spatially.

Coatings on devices have to be designed for mechanical stability and adhesion, especially when used in locations and device surfaces subject to substantial mechanical loads and/or friction. A particular example of such locations and surfaces are the joints of the skeletal system. Unfortunately, coatings having improved mechanical stability and adhesion may tend to have decreased utility as a controlled drug delivery vehicle.

U.S. Pat. No. 5,947,893 discloses a prosthesis having at least one porous tissue-mating surface, where the tissue-mating surface includes a coating having a pharmacologically active substance within a biodegradable carrier, such as a polymer or a biodegradable ceramic, such as calcium phosphate, wherein the biodegradable composition of the drug and carrier is impregnated within the pores of the tissue-mating surfaces of the device. Surface coatings, however, are vulnerable to mechanical failure and suffer other limitations. For instance, the choice of coating (drug formulation) material may be limited, because the material needs to be selected to yield a coating having sufficient structural integrity and adhesion properties. Moreover, thin coatings typically provide little actual control over the release kinetics of drugs, due to the extremely short diffusion path of drug from/through the coating. In addition, the use of a thicker coating can result in the creation of gaps between the prosthesis and the patient's tissue after the biodegradable matrix material of the drug formulation has degraded, which undesirably may permit differential motion between the prosthesis and adjacent tissue—and result in undesirable loosening of the prosthetic device. Furthermore, not all drugs are suitable for controlled release from a surface coating, for example, certain drugs, e.g., due to their high aqueous solubility, are released from the coatings at an undesirably high rate and cannot remain localized for a therapeutically effective amount of time. It would be desirable to provide devices and methods for controlling release kinetics of a variety of drugs from implantable prosthetic devices, while avoiding or substantially minimizing the limitations inherent in using a surface coating to modulate drug release.

### SUMMARY OF THE INVENTION

Improved implantable prosthetic devices are provided for controlled drug delivery. In one aspect, the device includes a prosthetic device body having at least one outer surface area; two or more discrete reservoirs located in spaced apart positions across at least a portion of the outer surface area, the reservoirs formed with an opening at the surface of the device body and extending into the device body; and a release system disposed in the reservoirs which comprises at least one therapeutic or prophylactic agent, wherein following implantation into a patient the therapeutic or prophylactic agent is released in a controlled manner from the reservoirs. The prosthetic device body preferably is a joint prosthesis or part thereof, such as a hip prosthesis, a knee prosthesis, a vertebral or spinal disc prosthesis, or part thereof. In another embodiment, the prosthetic device body comprises a surgical staple or surgical screw. In still another embodiment, the device body may comprise a dental implant or maxillofacial reconstruction device.

In various embodiments, the prosthetic device body and surface area in which the reservoirs are defined comprises a biocompatible material selected from metals, polymers, ceramics, and combinations thereof. In one embodiment, the device body comprises a stainless steel, a chrome-cobalt alloy, a titanium alloy, a ceramic, or an ultra high molecular weight polyethylene.

In a preferred embodiment, the prosthetic device body comprises a porous surface region, to promote tissue ingrowth. In one embodiment, the prosthetic device body further comprises a non-porous region, at least part of which is located beneath the porous region.

A variety of therapeutic or prophylactic agents can be delivered. In various embodiments, the therapeutic or prophylactic agent comprises an antibiotic agent, one or more growth factors, or a combination thereof. In one embodiment, the therapeutic or prophylactic agent is a self-propagating agent.

Release of the therapeutic or prophylactic agent preferably is passively controlled, but may be actively controlled in certain embodiments.

In various embodiments, the release system further includes one or more matrix materials. In one example, the matrix material comprises one or more synthetic polymers. In another example, the one or more matrix materials comprises a biodegradable, bioerodible, water-soluble, or water-swellable matrix material. In one embodiment, the therapeutic or prophylactic agent is distributed in the matrix material and the matrix material degrades or dissolves in vivo to controllably release the therapeutic or prophylactic agent. The therapeutic or prophylactic agent may be heterogeneously distributed in the reservoir or may be homogeneously distributed in the reservoir.

In a preferred embodiment, the one or more release system is provided in two or more layers having different compositions. In one example, each of the at least two reservoirs comprises at least two layers which comprise the one or more therapeutic or prophylactic agents and at least one layer of a degradable or dissolvable matrix material which does not comprise the one or more therapeutic or prophylactic agents. In another example, at least a first therapeutic or prophylactic agent is contained in a first layer of the two or more layers, and wherein a second therapeutic or prophylactic agent is contained in a second layer of the two or more layers.

Different therapeutic or prophylactic agents, or different doses, can be delivered from a single device, either from the same surface region or from different surface regions. In one embodiment, the quantity of therapeutic or prophylactic agent provided for release from at least a first of the reservoirs is different from the quantity of the therapeutic or prophylactic agent provided for release from at least a second of the reservoirs. In another embodiment, the time of release of one of the therapeutic or prophylactic agents from at least a first of the reservoirs is different from the time of release of the therapeutic or prophylactic agent from at least a second of the reservoirs. In one embodiment, a first therapeutic or prophylactic agent is in at least one of the reservoirs and a second therapeutic or prophylactic agent is in at least one other of the reservoirs, the first therapeutic or prophylactic agent and the second therapeutic or prophylactic agent being different in kind or dose.

In one embodiment, the device further includes one or more discrete reservoir caps positioned over or disposed in the reservoir openings, wherein the time and/or rate of release of the therapeutic or prophylactic agent is controlled by the reservoir caps. In a preferred embodiment, the reservoir caps are non-porous. In one embodiment, the reservoir caps have a thickness between 0.1 and 100 microns. In one embodiment for passive controlled release, the reservoir caps comprise a biodegradable or bioerodible polymer. For example, the biodegradable or bioerodible polymer may be selected from poly(lactic acids), poly(glycolic acids), poly(lactic-co-glycolic acids), poly(caprolactones), poly(anhydrides), and mixtures thereof. In one embodiment, at least one discrete reservoir cap is formed of a first material and at least one other discrete reservoir cap is formed of a second material, wherein the first material has a different degradation or dissolution rate compared to the second material. In another embodiment, at least one discrete reservoir cap has a first thickness and at least one other discrete reservoir cap has a second thickness, wherein the first thickness is different from the second thickness, thereby providing different times of release of the one or more therapeutic or prophylactic agent from the reservoirs covered respectively by the discrete reservoir cap having the first thickness and the discrete reservoir cap having the second thickness. In one embodiment for active controlled release, the reservoir caps comprise a metal film. In one embodiment, the device further includes control means to actively disintegrate the reservoir cap.

In preferred embodiments, the device includes arrays of many reservoirs, particularly an array of tens or hundreds of micro-reservoirs. In one embodiment, the device includes at least two rows of the at least two reservoirs. In one example, a first release system is in each of the at least two reservoirs of a first row and a second release system is in each of the at least two reservoirs of the other of the at least two rows other of the reservoirs, the first release system releasing the one or more therapeutic or prophylactic agents at a rate or in a dosage amount different from release of the one or more therapeutic or prophylactic agents from the second release system.

In a preferred embodiment, an implantable prosthetic device is provided for controlled drug delivery, which includes a prosthetic device body having at least one outer surface area; two or more discrete microreservoirs located in spaced apart positions across at least a portion of the outer surface area, the microreservoirs formed with an opening at the surface of the device body and extending into the device body; a release system disposed in the microreservoirs which comprises at least one therapeutic or prophylactic agent; and a plurality of discrete, non-porous reservoir caps located over the release system in the microreservoirs, wherein release of the therapeutic or prophylactic agent following implantation of the device into a patient is controlled by the reservoir caps. In a particular variation of this embodiment, the release system further comprises a matrix material, which further controls release of the therapeutic or prophylactic agent in vivo.

In another preferred embodiment, the implantable prosthetic device for controlled drug delivery includes a prosthetic device body having at least one outer surface area; two or more discrete microreservoirs located in spaced apart positions across at least a portion of the outer surface area, the microreservoirs formed with an opening at the surface of the device body and extending into the device body; and a release system disposed in the reservoirs which comprises at least one therapeutic or prophylactic agent and a biodegradable or bioerodible matrix material, wherein release of the therapeutic or prophylactic agent in vivo following implantation of the device into a patient is controlled by the matrix material. In a particular variation of this embodiment, the device further includes a plurality of discrete, non-porous reservoir caps located over the release system in the reservoir reservoirs, wherein the reservoir caps further control release of the therapeutic or prophylactic agent in vivo.

In another aspect, methods are provided for controllably releasing a therapeutic or prophylactic agent from a prosthetic device in vivo. In various embodiments, the method includes implanting the prosthetic devices described herein at a site in a patient; and releasing the therapeutic or prophylactic agent from the prosthetic device.

In one embodiment, a method is provided for local delivery of a therapeutic or prophylactic agent in the treatment of orthopedic tissues, such as joint spaces. In one case, the method includes implanting at a orthopedic tissue site, such as a joint or spinal disc, a tip portion of a tube which comprises a first end and a distal second end, wherein the tip portion has located therein a plurality of discrete reservoirs containing a therapeutic or prophylactic agent, the reservoirs having openings sealed by a plurality of discrete reservoir caps; and actively and selectively disintegrating the reservoir caps to initiate release of the therapeutic or prophylactic agent at the tissue site.

### BRIEF DESCRIPTION OF THE FIGURES

FIG. 1 is a perspective view and magnified view of one embodiment of hip prosthetic device that includes reservoirs for passive, controlled drug delivery.

FIGS. 2A-D are cross-sectional views of various embodiments of a prosthetic device body surface that includes regions of porosity and discrete reservoirs.

FIG. 3 is a plan view of one embodiment of a device which includes a tube which has a plurality of drug-containing reservoirs for active, controlled release of drug, for delivering drug into bone joints and other small spaces.

FIGS. 4A-C are plan (4A) and cross-sectional views (end on cross-section, 4B and side on cross-section, 4C) of one embodiment of the tip of the tube of the device shown in FIG. 3.

FIGS. 5A-C are perspective (5A) cross-sectional views (interior view 5B and end on cross-section, 5C) of one embodiment of a spinal cage prosthetic device.

FIG. 6 is a cross-sectional view of one embodiment of a knee prosthetic device that includes reservoirs for passive, controlled drug delivery.

FIGS. 7A-B are plan and cross-sectional perspective views of one embodiment of surgical screw that includes reservoirs for passive, controlled drug delivery.

FIGS. 8A-B are plan and cross-sectional perspective views of one embodiment of surgical staple that includes reservoirs for passive, controlled drug delivery.

FIG. 9 is a plan view of one embodiment of a spinal disc prosthetic device that includes reservoirs for passive, controlled drug delivery.

FIG. 10 is a cross-sectional view of one embodiment of a device implant comprising BMP-filled reservoirs adjacent bone tissue to facilitate tissue ingrowth.

### DETAILED DESCRIPTION OF THE INVENTION

Implantable orthopedic, spinal, and dental devices, in particular prosthetic joint and prosthetic disc devices, have been developed to provide improved controlled release of drug at the site of implantation. The released therapeutic or prophylactic agents are primarily intended for local or regional effect, but may in certain embodiments be intended for systemic delivery.

In one aspect, the device includes an array of discrete reservoirs (at least two and more preferably hundreds), particularly microreservoirs, that are provided across one or more outer surface areas of the device body. These reservoirs contain a release system comprising at least one therapeutic or prophylactic agent, and the release system and/or reservoir caps control the release kinetics (time and rate of release of the agent) in vivo following implantation. By containing the drug and controlled release formulation within discrete reservoirs built into (at least a portion of) the structure of the device body, one can avoid certain limitations that would otherwise have been obtained by use of a surface coating of the drug formulation, while enabling sustained or controlled drug release in complex temporal or spatial release profiles. For instance, one can use a desired drug formulation that might not have the mechanical strength properties needed for the drug formulation to be used as a surface coating on a prosthetic device body, but that works well when stored in discrete reservoirs located in a surface of the prosthetic device body.

As used herein, the term “orthopedic” includes and is synonymous with the term “orthopaedic.”

In another aspect, the “prosthetic” device body is a medical device primarily used to secure together separate tissue portions, or to provide a load bearing function. It is considered “prosthetic” in the sense that it is serving as a structural complement or substitute (permanently or temporarily) for one or more tissues of the body.

These devices can be used deliver a range of different drugs depending upon the particular application. In a preferred embodiment, the drug is used in the management of pain and swelling following the implantation surgery. For example, the device can release an effective amount of an analgesic agent alone or in combination with an anesthetic agent. In another embodiment, the drug helps minimize the risk of prosthetic joint infection or other site-specific infection due to implantation of an orthopedic or dental device. For example, the device can release a therapeutic or prophylactic effective amount one or more antibiotics (e.g., cefazolin, cephalosporin, tobramycin, gentamycin, etc.) and/or another agent effective in preventing or mitigating biofilms (e.g., a quorum-sensing blocker or other agent targeting biofilm integrity). Bacteria tend to form biofilms on the surface of implant devices, and these biofilms, which are essentially a microbial ecosystem with a protective barrier, are relatively impermeable to antibiotics. Accordingly, systemically administered antibiotics may not achieve optimal dosing where it is needed most. However, the present devices enable the delivery of the desired dose of antibiotic precisely when and precisely where needed—in particular beneath the biofilm. In addition, the device can be designed to release the drug in various temporal and spatial patterns/profiles, e.g., releasing drug in a continuous or pulsatile manner for several (e.g., 5 to 15) days and/or targeting areas of the device, if any, that are more conducive to bacterial growth. In one embodiment, revision implants are provided with reservoirs on the implant surface or in crevices or channels, which are loaded with a stable antibiotic formulation with optimized release kinetics. In this way, the antibiotic agent can be released under a bacterial biofilm that may form from bacteria harbored in crevices of a prosthetic implant. The local delivery of antibiotic agents can decrease undesirable systemic drug exposure (and deleterious side effects caused thereby). In another embodiment, following a total knee replacement, the prosthetic knee device includes a plurality of discrete reservoirs for releasing an antibiotic or other drug.

In a preferred embodiment, the present drug-eluting device is adapted for use in the treatment of cancer of the bone or joint. For example, osteosarcoma or chondrosarcoma often are treated surgically by excision requiring removal of significant amounts of bone and soft tissue. Care must be taken to avoid spilling the tumor during resection to avoid seeding of tumor cells into surrounding tissues. It therefore would be beneficial for the prosthetic implant to release one or more local chemotherapeutic agents into the surrounding tissue following implantation, in order to destroy tumor cells remaining at the surgical site following resection, to complement or replace the systemic chemotherapy and/or radiation therapy that typically is prescribed for the patient. In variations of these embodiments, the implant device releases one or a combination of therapeutic agents, including chemotherapeutic agents (e.g., paclitaxel, vincristine, ifosfamide, dacttinomycin, doxorubicin, cyclophosphamide, and the like), bisphosphonates (e.g., alendronate, pamidronate, clodronate, zoledronic acid, and ibandronic acid), analgesics (such as opoids and NSAIDS), anesthetics (e.g., ketoamine, bupivacaine and ropivacaine), tramadol, and dexamethasone.

In another embodiment, the drug facilitates vascularization at or into the implanted prosthetic device or promotes bone health and growth. For example, the drug can be a bone morphogenic protein (BMP) or recombinant version thereof (rBMP), which facilitates bone formation around or, in the case of a device having a porous surface, into the implanted prosthetic device. Representative examples of BMPs include BMP-2, -3, -4, -7, -9, and -13; others known in the art may also be used. In one embodiment, the drug is rhBMP-2. In one example for spinal ligament repair, the drug is BMP-13. The use of a prosthetic device with a drug or drugs that facilitates vascularization and/or promotes bone health and growth may be particularly desirable where the prosthesis is secured without the use of cement, although it could possibly be used in combination with a cement.

In several preferred embodiments, the device releases a combination of different substances to improve bone healing. For example, the device can release different combinations of growth factors (e.g., (TGF)-β, BMP, OP-1, MP52, VEGF), osteoinductive molecules, hormones, anti-TNF (tumor necrosis factor) agents, and bone-forming cells (e.g., osteoblasts, adult stem cells, osteoprogenitor cells). These different molecules and cells can be delivered at varied spatial positions and temporal sequences during bone healing. In one particular embodiment for the repair of local bone erosions, which often are associated with rheumatoid arthritis, the prosthetic device locally delivers (1) an anti-TNF agent, which reduces inflammation that fuels bone erosion, and (2) parathyroid hormone (PTH), which stimulates bone formation, and/or osteoprotegrin (OPG), which blocks bone resorption and can lead to repair of local bone erosions and reversal of systemic bone loss. Examples of anti-TNF agents include TNF antagonists, such as etanercept (Enbrel™, Amgen and Wyeth) and infliximab (Remicade™, Centocor), which have shown efficacy and have been approved by the U.S. FDA for the treatment of rheumatoid arthritis.

In yet another embodiment, the drug can be one selected to mitigate the risk of formation of blood clots at the implant site, which can lead to venous thromboembolism or pulmonary embolism. For instance, the device may be used to release one or more anticoagulants and/or antiplatlet drugs (e.g., heparins, aspirin, clopidogrel, lepirudin, fondaparinux, warfarins, dicumarol, etc.).

In still a further embodiment, the drug stored in and released from the reservoirs is a self-propagating agent, such as a gene therapy agent or vector. A desired local or systemic response is created following release of the small amount of agent.

Representative examples of therapeutic or prophylactic agents that may be released from the prosthetic device include analgesics, anesthetics, antimicrobial agents, antibodies, anticoagulants, antifibrinolytic agents, anti-inflammatory agents, antiparasitic agents, antiviral agents, cytokines, cytotoxins or cell proliferation inhibiting agents, chemotherapeutic agents, hormones, interferons, and combinations thereof. In one embodiment, the device provides for the controlled release of a growth factor, such fibroblast growth factors, platelet-derived growth factors, insulin-like growth factors, epidermal growth factors, transforming growth factors, cartilage-inducing factors, osteoid-inducing factors, osteogenin and other bone growth factors, and collagen growth factors. In another embodiment, the device provides for controlled release of a neutrophic factor (which may be of benefit in spinal prosthetic applications) or a neutropic factor. In one embodiment, the drug is a tumor necrosis factor.

In one embodiment, the drug is in an encapsulated form. For example, the drug can be provided in microspheres or liposomes for sustained release.

Preferably, release of drug is passively controlled. However, the prosthetic device body can include active mechanisms for controlling release from reservoirs, as detailed below. The active control and/or power mechanisms could, for example, be attached to or imbedded within a surface of the prosthetic device, or could be built into inside (e.g., in an interior space of) the prosthetic device.

### ILLUSTRATIVE EMBODIMENTS OF THE DEVICES

In one aspect, an implantable prosthetic device for controlled drug delivery is provided which includes: a prosthetic device body having at least one outer surface area; two or more discrete reservoirs located in spaced apart positions across at least a portion of the outer surface area, the reservoirs being formed with an opening at the surface of the device body and extending into the device body; a release system disposed in the reservoirs which comprises at least one therapeutic or prophylactic agent, wherein following implantation into a patient the therapeutic or prophylactic agent is released in a controlled manner, at effective rates/times, from the reservoirs.

Device Body

The device body is substantially rigid, with a defined geometry. That is, it is not a spongy or putty-like material that takes the shape of the space in which it is implanted.

In one embodiment, the prosthetic device body is a joint or bone prosthesis or part thereof. Examples of typical prosthetic joints include knees, hips, shoulders, and to a lesser extent, elbow, wrist, ankle, and finger joints. In a preferred embodiment, the bone prosthesis is adapted for use in a knee replacement, or a hip replacement. The hip is essentially a ball and socket joint, linking the “ball” at the head of the thigh bone (femur) with the cup-shaped “socket” in the pelvic bone. A total hip prosthesis is surgically implanted to replace the damaged bone within the hip joint. In one example, the total hip prosthesis consists of three parts: (1) a metal cup (called the acetabulum or acetabular component) that replaces the hip socket, which cup has a liner made of a polymer (e.g., a high molecular weight polyethylene), ceramic, or metal material; (2) a metal or ceramic ball that replaces the damaged head of the femur; and (3) a metal stem that is inserted into or attached to the shaft of the bone to add stability to the prosthesis. The reservoirs can be provided on any or all surfaces of such a prosthesis.

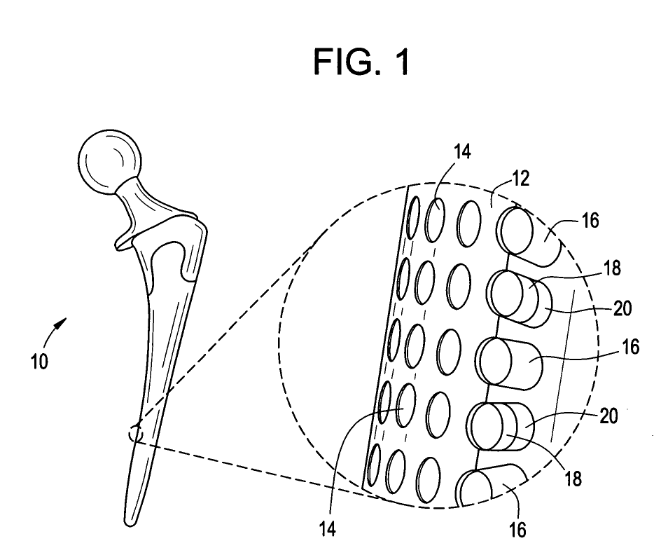

One embodiment of a hip prosthetic device is shown in FIG. 1, which illustrates the ball and stem portion 10 of a hip prosthesis. The device body has an outer surface 12 which includes an array of reservoirs 14 disposed therein. In this particular embodiment, select reservoirs contain a first drug formulation 16, and select other reservoirs contain a second drug formulation 20. The reservoirs of the second drug formulation include reservoir caps 18 covering the second drug formulation 20.

In other embodiments, the bone prosthesis may be another joint prosthesis, such as a knee, shoulder, or elbow. In still other embodiments, the bone prosthesis may be a spinal disc, a spinal cage, or a dental implant.

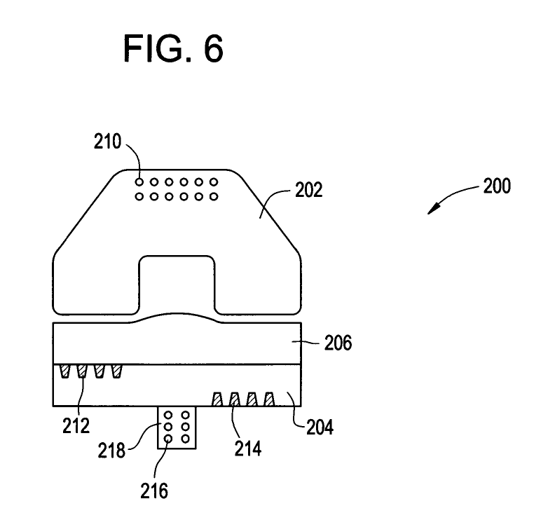

One embodiment of a knee prosthetic device is shown in FIG. 6. Device 200 includes a tibial base plate 204, a polyethylene component 206, and a metal femoral component 202, for use in a total knee arthroplasty (TKA). In this particular embodiment, the various outer surfaces of the device body include arrays of reservoirs disposed therein. Reservoirs 210 contain a first drug formulation to be released from femoral component 202. Reservoirs 212 contain a second drug formulation (e.g., an antibiotic) and 214 contains a third drug formulation (e.g., a growth factor) to be released from tibial base plate 204. Reservoirs 216 contain a fourth drug formulation (e.g., a growth factor) to be released from the anchor portion 218 of tibial base plate 204.

In one embodiment, the device is a spinal disc prosthesis. For example, it could be an adaptation of, or similar to, the FDA-approved CHARITÉ™ (DePuy Spine, Inc., Raynham, Mass.) disc which comprises cobalt chromium endplates and an ultra-high molecular weight polyethylene (UHMWPE) sliding core. In one example, the endplates are provided with an array of discrete reservoirs in one or more surfaces, which are loaded with a release system comprising one or more therapeutic or prophylactic agents for controlled release. In another embodiment, the device is a spinal infusion device, such as a modification of the INFUSE™ Bone Graft/LT-CAGE™ (Medtronic Inc.) lumbar tapered fusion device, which is indicated for spinal fusion procedures in skeletally mature patients with degenerative disc disease (DDD). In one modification, the device body, or cage, that holds the rBMP-soaked sponge is itself provided a plurality of reservoirs, for releasing one or more bioactive agents, to enhance to effectiveness of the device. For instance, the reservoirs could release additional rBMP, antibiotics, analgesics, anesthetics, or combinations thereof. In another variation, the cage device is modified so that the separate rBMP-soaked sponge is no longer needed, thereby greatly simplifying the device preparation steps preceding implantation. For example, the cage device itself can be modified to include reservoirs on the inside and/or outside walls of the cage. These reservoirs contain and passively release an rBMP formulation. As for providing a tissue scaffold or other osteoconductive material inside the cage, the interior can include a dry hydrogel coating material. The surgeon simply wets the coating with saline prior to implantation of the device—no longer need to prepare solution, soak the sponges, and then insert the sponges into the cage. Furthermore, the interior of the cage can be made to have a series of baffles to provide additional surface area for bone growth and/or additional surface area for drug-containing reservoirs.

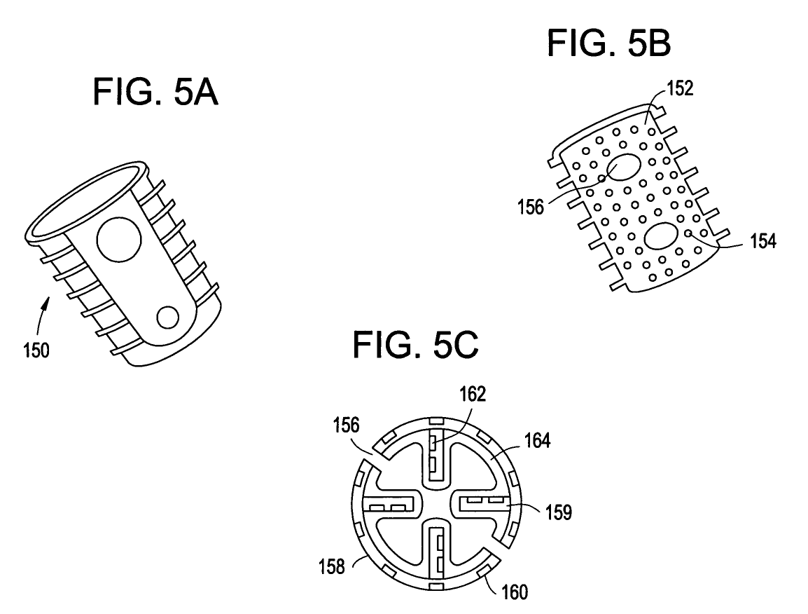

One embodiment of a lumbar tapered fusion prosthetic device is shown in FIGS. 5A-C. Device 150 includes interior surface 152 in which interior reservoirs 154 are disposed. The device body includes sidewall 158 which has exterior reservoirs 160 and major apertures 156, which are provided for bone to grow into/through the device to lock it into place, providing a bridge of bone extending from one vertebrae to the next. The interior of the device includes baffles 159, which are coated with a tissue scaffolding material 164, such as a hydrogel. The baffles also include baffle reservoirs 162.

In another embodiment, the device is for disc and vertebral replacement. For example, the device can be an artificial disc similar to the MAVERICK™ (Medtronic Sofamor Danek) artificial disc for use in patients who suffer from degenerative disc disease. In a further embodiment, the device is used in the treatment of ankylosing spondylitis, a rheumatic disease characterized by inflammation of joints and ligaments, which results in bone erosion, most often in the spine but sometimes in other joints as well. The formation of new bone during healing can lead to the fusing of vertebrae and spine rigidity. The device preferably is provided with a plurality of discrete reservoirs, which can be located for example in screws of the device and in surfaces contacting the vertebrae. Such reservoirs could be loaded with a stable formulation of a bone growth factor with optimised release kinetics and optionally loaded with an antibiotic agent for biofilm control. These or other reservoirs could be sized and located to enhance device fixation, e.g., by promoting osteointegration.

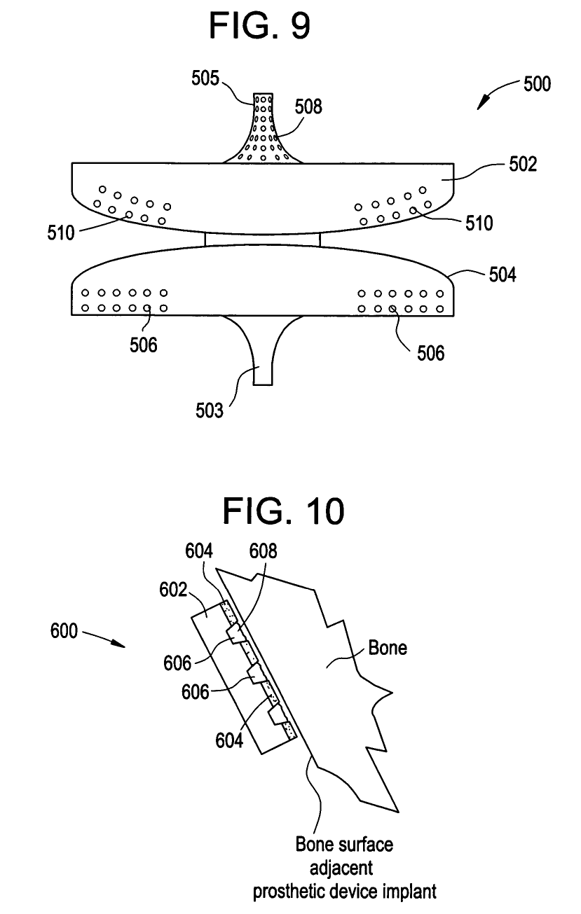

One embodiment of artificial disc is shown in FIG. 9. Device 500 includes an upper disc component 502 and a lower disc component 504. The upper disc component includes anchor portion 505, the surface of which includes an array of reservoirs 508 disposed therein. The lower disc component includes anchor portion 503. Upper disc component 502 includes reservoirs 510. Lower disc component 504 includes reservoirs 506.

In still other embodiments, the device is a dental or maxillofacial prosthetic device. For instance, a maxillofacial prosthetic device may be desirable in reconstructive surgery needed to repair a traumatic facial injury or congenital defect. It may be in the form of a plate, which can be screwed into existing bone. In a preferred variation, the reservoirs of the device release one or more anti-infective agents. In one embodiment, the dental prosthetic device includes an anchor portion for anchoring in a bone structure and a head intended to support a dental prosthesis, and reservoirs are provided in one or more parts, preferably at the anchor portion. Typically, the reservoirs deliver one or more drugs locally at the implant site over an extended period of time following implantation. Other dental prosthesis known in the art can be adapted to include the reservoir-based controlled release formulations described herein. See U.S. Pat. No. 6,799,970, which is expressly incorporated herein by reference.

In yet another embodiment, the device body is a surgical staple or a surgical screw. The staple or screw is provided with a plurality of microreservoirs that store and release drug. In one embodiment, the staple or screw is biodegradable and releases the drug in a defined manner as the screw or staple degrades. In another embodiment, the screw or staple is non-biodegradable, and the plurality of microreservoirs located in the surface of the screw or staple release drug in a defined manner, as dictated by the structure of the reservoir (shape, size, etc.) and the particular drug formulation contained in the reservoirs. Representative examples of screws and staples that could be modified to include drug containing and releasing reservoirs are described in U.S. Pat. No. 5,961,521 to Roger, which is expressly incorporated herein by reference.

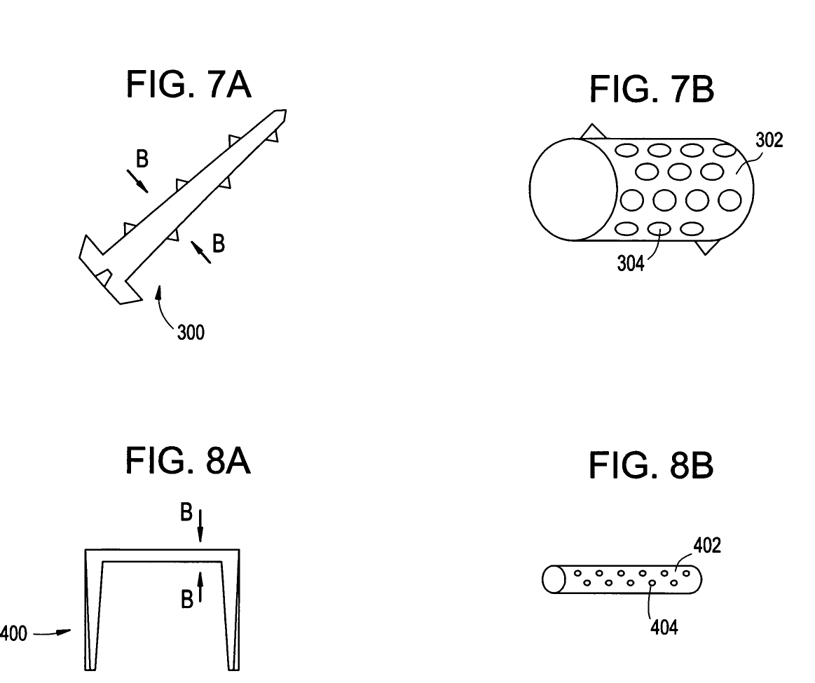

FIGS. 7A-B illustrate a surgical screw 300 which has an outer surface 302 in which a plurality of reservoirs 304 are disposed. FIG. 7B is a close up sectional view of part of the surgical screw 300 of FIG. 7A.

FIGS. 8A-B illustrate a surgical staple 400 which has an outer surface 402 in which a plurality of reservoirs 404 are disposed. FIG. 8B is a close up sectional view of part of the surgical staple 400 of FIG. 8A.

In preferred embodiments, the device body and surface area in which the reservoirs are defined can be formed of, be coated with, or otherwise comprise a biocompatible material selected from metals, polymers, ceramics, and combinations thereof. Typically, the device body is non-biodegradable, as the prosthetic device is intended to last for an extended period of time, preferably for the life of the patient. For instance, the device body can comprise a stainless steel, a chrome-cobalt alloy, a titanium alloy, a ceramic, or an ultra high molecular weight polyethylene (e.g., a highly cross-linked, UHMW polyethylene). In other embodiments, the device body is formed of or includes a ceramic (e.g., alumina, silicon nitride, zirconium oxide), a semiconductor (e.g., silicon), a glass (e.g., Pyrex™, BPSG), or a degradable or non-degradable polymer.

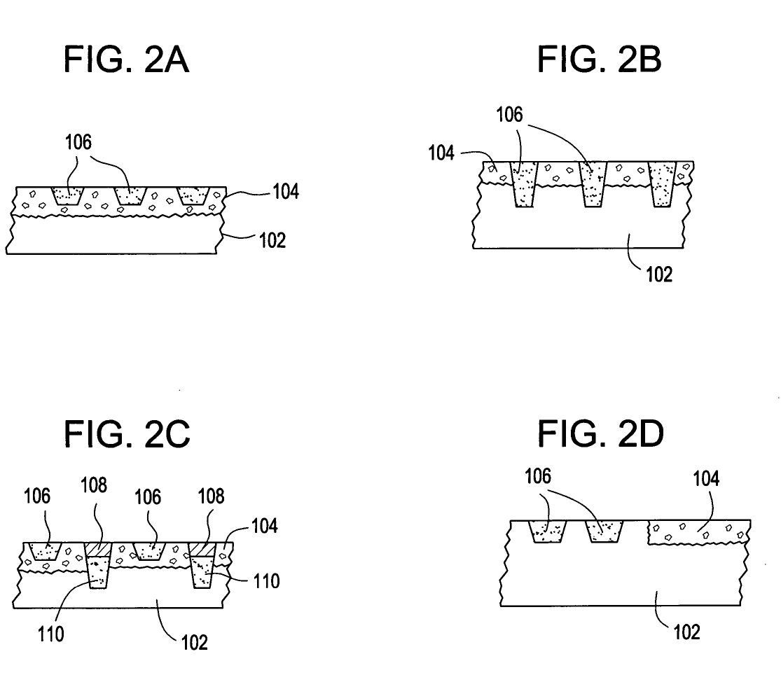

The surface of the device body where the reservoirs are located can be porous or non-porous. Optimal bony-ingrowth is expected to be provided into prosthesis devices that include pores of approximately 250 to 500 microns. In one embodiment, the entire surface of the device is porous. In another embodiment, a portion, e.g., a portion of the tissue- or bone-mating surfaces, of the prosthesis is porous, to provide at least one tissue-contact surface that provides stable fixation in the body. FIGS. 2A-C illustrate, in cross-sectional views, some of the various combinations of porous and non-porous substrate (body) materials with different reservoirs. FIG. 2A shows a portion of a device body having non-porous region 102 with porous surface region 104, in which discrete reservoirs are disposed in spaced positions (i.e., in an array). The reservoirs are filled with drug formulation 106, such as drug dispersed in a soluble or biodegradable matrix material, such as biocompatible polymer, e.g., PLGA, PEG, or various poly(anhydrides). In this embodiment, the reservoirs are located only in the porous region. In contrast, FIG. 2B show a device in which the reservoirs extend into the non-porous region. In FIG. 2C, some reservoirs are shallower and some are deeper, such that only the deeper ones extend into the non-porous region. In this embodiment, the shallower reservoirs contain a first drug formulation 106, and the deeper reservoirs are filled with two or more distinct layers: An outer layer 108, which can be formed of one or more non-bioactive materials (e.g., a biodegradable, protective reservoir cap) that can delay exposure of an inner layer 110, which can comprise a drug—the same as or different from the drug in formulation 106. FIG. 2D illustrates an embodiment having a surface comprising both porous and non-porous regions. The non-porous region 102 includes reservoirs containing drug formulation 106, and the porous region 104 may, for example, be selected to have a porosity that facilitates tissue ingrowth. In one embodiment, the device body includes or consists of a completely porous material, such as a trabecular metal, e.g., tantalum (provided by Zimmer, Inc.). Other variations and combinations of these embodiments are envisioned.

In one particular embodiment, tissue ingrowth into the prosthetic implant can be enhanced through the use of reservoirs, preferably macro-reservoirs, containing a bone morphogenic protein, optionally with a calcium phosphate compound, at an implant surface that is placed adjacent the bone (particularly “bleeding bone”) site at which tissue growth is desired. The implant surface preferably is porous and optionally may itself include a coating of therapeutic or prophylactic compound (e.g., the same bone morphogenic protein, calcium phosphate, or combination thereof in the reservoirs). This embodiment is based on the observation that a bone defect can induce bone ingrowth, as described in Bragdon, et al., Clinical Orthopaedics & Related Research, 417: 50-61 (2003). Here, the reservoirs effectively act as a defect, i.e., an area of non-contact between the implant surface and the bone tissue surface. One embodiment is illustrated in FIG. 10, which shows device 600, which comprises substrate 602, porous surface 604, and reservoirs 606. The reservoirs, and optionally the pores in porous surface 604, are filled with a BMP formulation 608.

Optionally, the device body may be installed into the bone site with a biocompatible cement. The surface of the device body to be cemented can be porous or non-porous. Examples of biocompatible cements known in the art include polymethylmethacrylates (PMMAs) and PALACOS™ (Heraeus Kulzer, Germany).

The shape of the device body depends on the particular application. The device body preferably is a non-degradable structure. The body may consist of only one material, or may be a composite or multi-laminate material, that is, composed of several layers of the same or different substrate materials that are bonded together.

In another embodiment, the device body is not actually a prosthetic but is used in the treatment of an orthopedic disease or disorder. In one embodiment, a method is provided for local delivery of a therapeutic or prophylactic agent in the treatment of difficult to access orthopedic tissues, such as joint spaces. This is particularly wherein active control of drug release is desired, but there is little or no space for a larger implant device with associated electronics and power sources. In one case, the method includes implanting at a orthopedic tissue site, such as a joint or spinal disc, a tip portion of a tube which comprises a first end and a distal second end, wherein the tip portion has located therein a plurality of discrete reservoirs containing a therapeutic or prophylactic agent, the reservoirs having openings sealed by a plurality of discrete reservoir caps; and actively and selectively disintegrating the reservoir caps to initiate release of the therapeutic or prophylactic agent at the tissue site. The reservoir caps are electrically connected to a power source and can be disintegrated by electrothermal ablation as described in U.S. patent application Publication No. 2004/0121486 A1, which is incorporated herein by reference. Preferably, the tip includes tens or hundreds of micro-reservoirs containing a drug formulation and hermetically sealed by conductive reservoir caps. The tube tip can be made of biocompatible metal, ceramic, silicon, or polymer, and it serves as the substrate in which the discrete reservoirs are fabricated and arrayed. The power source and control hardware can be surgically placed in a subcutaneous pocket under the intraclavicular fossa or in the abdominal wall, and the tube extending therefrom can be threaded into the therapeutically desirable location at a vertebrae. The device is able to store and release anti-TNF agent as needed at precise dosages over an extended period of time. In one embodiment, the tube portion is replaceable and removably secured to the power/control unit, so that when all of the reservoirs are depleted of drug, then the tube can be replaced with a minimally invasive procedure, since the power/control unit need not be replaced as frequently, if at all. The implanted power/control unit can be battery powered and pre-programmed or wirelessly powered and wirelessly controlled externally. The tip also may be placed in or near joints where a larger device could not fit. For example, the tip may be placed in the intercondylar fossa in the knee joint to release anti-infectives or anti-inflammatory drugs. The power source and control electronics could be placed under the skin in the thigh or in the abdomen.

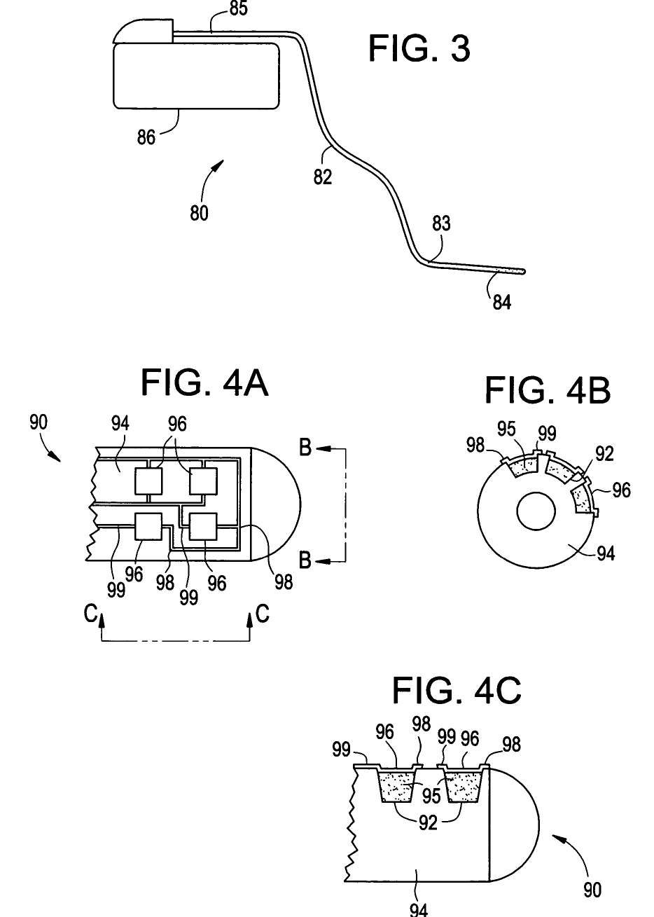

In one embodiment, which is illustrated in FIG. 3, the implantable device 80 includes a catheter or tube 82 which has a plurality of drug-containing reservoirs 84 fabricated at the tip portion 83 of the tube. The power source and control hardware 86 are located at the proximal end of the tube 85, so they need not fit into or be located at the delivery site. Alternatively, the power/control unit can be externally worn and provided with a tube through the patient's skin. The electrical traces could be built into the tube body or supported on an inner or outer surface of the tube body. FIGS. 4A-C illustrates one embodiment of the tube tip portion 90 which has reservoirs 92 in substrate/ tube body 94, wherein the reservoirs contain therapeutic agent 95 and are covered by conductive reservoir caps 96, each of which are connected to input and output electrical leads 98 and 99, respectively.

In one application, the device is intended for the treatment of ankylosing spondylitis (AS). In a preferred embodiment, the drug formulation comprises an anti-TNF-α monoclonal antibody. In use, the tip portion is inserted next to or attached to a vertebrae of an AS patient, to locally release the drug formulation at the bone or near the disc.

In another aspect, a flexible drug delivery device is provided for wrapping around bone tissue. In one embodiment, the device includes a flexible (e.g., polymeric) film that contains discrete pellets (effectively reservoirs) a therapeutic or prophylactic agent formulation. The pellets could, for example, be laminated between two layers of polymeric sheets to trap and contain the drug. The sheets could be porous and/or biodegradable. The sheets could be “shrink-wrappable” around the bone to tightly conform to the bone tissue surface. To facilitate implantation, the device could be provided in the form of strips that could be manually wrapped around bone tissue areas. In one embodiment, the drug may contain an anti-infective agent.

Reservoirs

The reservoirs are located in spaced apart positions across one or more areas of the surface of the device body. The reservoirs are formed with an opening at the surface of the device body and extend into, or through, the device body. In preferred embodiments, the reservoirs are discrete, non-deformable, and disposed in an array across one or more surfaces (or areas thereof) of the device body. As used herein, the term “reservoir” means a well, a cavity, or a hole suitable for storing, containing, and releasing/exposing a precise quantity of a material, such as a drug formulation. The interconnected pores of a porous material are not reservoirs. Pores are not considered reservoirs, because of their random nature (random in size, shape, and location), which renders them unsuitable for controlling release kinetics. That is, one cannot accurately know the amount of drug contained within a porous material, the control of the release kinetics is much more difficult.

Reservoirs can be created in the device body at the simultaneously with formation of the device body, or it can be made formed in the device body after the device body is made. Accordingly, the reservoirs can be made by a variety of techniques, including MEMS fabrication processes, microfabrication processes, or other micromachining processes, various drilling techniques (e.g., laser, mechanical, and ultrasonic drilling), build-up or lamination techniques, such as LTCC (low temperature co-fired ceramics), and sand, grit, and other particle blasting techniques. Numerous other methods known in the art can also be used to form the reservoirs. See, for example, U.S. Pat. No. 6,123,861 and U.S. Pat. No. 6,808,522. Microfabrication methods include lithography and etching, injection molding and hot embossing, electroforming/electroplating, microdrilling (e.g., laser drilling), micromilling, electrical discharge machining (EDM), photopolymerization, surface micromachining, high-aspect ratio methods (e.g., LIGA), micro stereo lithography, silicon micromachining, rapid prototyping, and DEEMO (Dry Etching, Electroplating, Molding).

The reservoirs can be fabricated into the device body by any of a number of methods and techniques known in the art, depending on various parameters including the materials of construction of the device body, the dimensions of the reservoirs, the location of the reservoirs on the device body, and the shape and configuration of the device body. In one embodiment, the reservoirs are formed in the substrate by laser drilling, EDM, or other mechanical or physical ablative methods. In another embodiment, the reservoirs are fabricated by a masking and chemical etching process. In embodiments where the device comprises a porous surface, the reservoirs can be fabricated before or after a porosity-inducing step. For instance, reservoirs can be mechanically formed into the porous surface, optionally penetrating into the non-porous region beneath. Alternatively, porosity can be creating in the surface, for example, by a chemical etching process after formation of the reservoirs. In order to preserve the defined boundaries of the reservoirs, the reservoirs can be filled with a temporary fill material, such as a wax, that is resistant to the chemical etch, prior to etching and then the fill material can be removed following etching, for example, by heating and volatilizing the wax or by use of an appropriate solvent selective for the temporary fill material. One process for creating surface microporosity in a titanium or other metal surface is described in U.S. patent application Publication No. 2003/0108659 A1 to Bales et al., which is incorporated herein by reference.

The device body preferably has many reservoirs. In various embodiments, tens, hundreds, or thousands of reservoirs are arrayed across the device body.

The reservoirs may be defined by one or more sidewalls, a bottom wall, an open end (an opening) distal the bottom wall. The opening is at a surface of the device body from which release of the therapeutic or prophylactic agent is desired. In a preferred embodiment, all of the reservoir walls (side and bottom) are non-porous. In another embodiment, a majority of the reservoir walls are non-porous, e.g., where the reservoir extends through a porous surface region (and into a non-porous region) of the device body. In another embodiment, reservoirs may extend through the device body, providing for instance a reservoir having two opposed openings (no bottom wall).

In a preferred embodiment, the reservoirs are microreservoirs. The use of microreservoirs may be particularly beneficial to minimally impact the strength and structural integrity of the device body, as compared to the mechanical property losses that could occur with the use of macroreservoirs. As used herein, the term “microreservoir” is a reservoir having a volume equal to or less than 500 μL (e.g., less than 250 μL, less than 100 μL, less than 50 μL, less than 25 μL, less than 10 μL, etc.) and greater than about 1 nL (e.g., greater than 5 nL, greater than 10 nL, greater than about 25 nL, greater than about 50 nL, greater than about 1 μL, etc.). In certain embodiments, the reservoirs are macroreservoirs. A “macroreservoir” is a reservoir having a volume greater than 500 μL (e.g., greater than 600 μL, greater than 750 μL, greater than 900 μL, greater than 1 mL, etc.) and less than 5 mL (e.g., less than 4 mL, less than 3 mL, less than 2 mL, less than 1 mL, etc.). The shape and dimensions of the reservoir, as well as the number of reservoirs, can be selected to control the contact area between the drug material and the surrounding surface of the reservoirs. Unless explicitly indicated to be limited to either micro- or macro-scale volumes/quantities, the term “reservoir” is intended to encompass both.

Release System and Therapeutic/Prophylactic Agent

The release system comprises at least one therapeutic or prophylactic agent (sometimes referred to herein as a “drug”). The release system is disposed in the reservoirs, so as to be isolated, e.g., protected, from the environment outside of the reservoir until a selected point in time, when its release or exposure is desired. The term “release system,” is as described in U.S. Pat. No. 5,797,898, which is incorporated herein by reference. The therapeutic or prophylactic agent can be dispersed in a matrix material, which by its degradation, dissolution, or diffusion properties provides a means for controlling the release kinetics of the therapeutic or prophylactic agent.

The therapeutic or prophylactic agent can be essentially any active pharmaceutical ingredient, or API. It can be natural or synthetic, organic or inorganic molecules or mixtures thereof. The therapeutic or prophylactic agent molecules can be mixed with other materials to control or enhance the rate and/or time of release from an opened reservoir.

The therapeutic or prophylactic agent molecules may be in essentially any form, such as a pure solid or liquid, a gel or hydrogel, a solution, an emulsion, a dispersion, a slurry, or a suspension. In various embodiments, the therapeutic or prophylactic agent molecules may be in the form of solid mixtures, including amorphous and crystalline mixed powders, monolithic solid mixtures, lyophilized powders, and solid interpenetrating networks. In other embodiments, the molecules are in liquid-comprising forms, such as solutions, emulsions, colloidal suspensions, slurries, or gel mixtures such as hydrogels.

In a preferred embodiment, the drug is provided in a solid form, particularly for purposes of maintaining or extending the stability of the drug over a commercially and medically useful time, e.g., during storage in a drug delivery device until the drug needs to be administered. The solid drug matrix may be in pure form or in the form of solid particles of another material in which the drug is contained, suspended, or dispersed. In one embodiment, the drug is formulated with an excipient material that is useful for accelerating release, e.g., a water-swellable material that can aid in pushing the drug out of the reservoir and through any tissue capsule over the reservoir.

In one embodiment, the drug is formulated with one or more excipients that facilitate transport through tissue capsules. Examples of such excipients include solvents such as DMSO or collagen- or fibrin-degrading enzymes.

The drug can comprise small molecules, large (i.e., macro-) molecules, or a combination thereof. In one embodiment, the large molecule drug is a protein or a peptide. Representative examples of drugs and drug types include amino acids, vaccines, antiviral agents, gene delivery vectors, interleukin inhibitors, immunomodulators, neurotropic factors, neuroprotective agents, antineoplastic agents, chemotherapeutic agents, polysaccharides, anti-coagulants (e.g., LMWH, pentasaccharides), antibiotics (e.g., immunosuppressants), analgesic agents, and vitamins. In one embodiment, the drug is a protein. Examples of suitable types of proteins include, glycoproteins, enzymes (e.g., proteolytic enzymes), hormones or other analogs (e.g., LHRH, steroids, corticosteroids, growth factors), antibodies (e.g., anti-VEGF antibodies, tumor necrosis factor inhibitors), cytokines (e.g., α-, β-, or γ-interferons), interleukins (e.g., IL-2, IL-10), and diabetes/obesity-related therapeutics (e.g., insulin, exenatide, PYY, GLP-1 and its analogs). In one embodiment, the drug is a gonadotropin-releasing (LHRH) hormone analog, such as leuprolide. In another exemplary embodiment, the drug comprises parathyroid hormone, such as a human parathyroid hormone or its analogs, e.g., hPTH(1-84) or hPTH(1-34). In a further embodiment, the drug is selected from nucleosides, nucleotides, and analogs and conjugates thereof. In yet another embodiment, the drug comprises a peptide with natriuretic activity, such as atrial natriuretic peptide (ANP), B-type (or brain) natriuretic peptide (BNP), C-type natriuretic peptide (CNP), or dendroaspis natriuretic peptide (DNP). In still another embodiment, the drug is selected from diuretics, vasodilators, inotropic agents, anti-arrhythmic agents, Ca⁺ channel blocking agents, anti-adrenergics/sympatholytics, and renin angiotensin system antagonists. In one embodiment, the drug is a VEGF inhibitor, VEGF antibody, VEGF antibody fragment, or another anti-angiogenic agent. In yet a further embodiment, the drug is a prostaglandin, a prostacyclin, or another drug effective in the treatment of peripheral vascular disease. In still another embodiment, the drug is an angiogenic agent, such as VEGF. In a further embodiment, the drug is an anti-inflammatory, such as dexamethasone. In one embodiment, a device includes both angiogenic agents and anti-inflammatory agents. In still another embodiment, the drug is a growth factor known in the art for chondrogenesis, including fibroblast growth factor (FGF), insulin-like growth factor (IGF), transforming growth factor beta (TGB-β)

The reservoirs in one device can include a single drug or a combination of two or more drugs, and can further include one or more pharmaceutically acceptable carriers. Two or more drugs can be stored together and released from the same one or more reservoirs or they can each be stored in and released from different reservoirs.

The release system may include one or more pharmaceutical excipients. The release system may provide a temporally modulated release profile (e.g., pulsatile release) when time variation in plasma levels is desired or a more continuous or consistent release profile when a constant plasma level as needed to enhance a therapeutic effect, for example. Pulsatile release can be achieved from an individual reservoir, from a plurality of reservoirs, or a combination thereof. For example, where each reservoir provides only a single pulse, multiple pulses (i.e., pulsatile release) are achieved by temporally staggering the single pulse release from each of several reservoirs. Alternatively, multiple pulses can be achieved from a single reservoir by incorporating several layers of a release system and other materials into a single reservoir. Continuous release can be achieved by incorporating a release system that degrades, dissolves, or allows diffusion of molecules through it over an extended period. In addition, continuous release can be approximated by releasing several pulses of molecules in rapid succession (“digital” release). The active release systems described herein can be used alone or on combination with passive release systems, for example, as described in U.S. Pat. No. 5,797,898. For example, the reservoir cap can be removed by active means to expose a passive release system, or a given substrate can include both passive and active release reservoirs.

In one embodiment, the drug formulation within a reservoir comprises layers of drug and non-drug material. After the active release mechanism has exposed the reservoir contents, the multiple layers provide multiple pulses of drug release due to intervening layers of non-drug. In another embodiment, multiple layers having different compositions are used, and the different layers all contain a drug, which can be the same or different among the layers. In another embodiment, different surface areas or parts of the prosthetic implant device can have different numbers, sizes, and densities of reservoirs from other areas or parts of the device, and different reservoirs can be loaded with different drugs and/or different formulations have different release kinetics from other reservoirs. Such various strategies can be used to obtain complex release profiles in a single device, tailored for a particular indication or patient.

Reservoir Caps

In an optional embodiment, the device further includes reservoir caps. A reservoir cap is a discrete structure (e.g., a membrane or thin film) positioned over or disposed in (thereby blocking) the opening of a reservoir to separate the (other) contents of the reservoir from the environment outside of the reservoir. It controls, alone or in combination with the release system, the time and/or rate of release of the therapeutic or prophylactic agent from the reservoir. For example, release can be controlled by selecting which reservoir caps, how many reservoir caps, and at what time the reservoir caps are disintegrated or made permeable.

In a preferred embodiment, the reservoir caps are non-porous and are formed of a bioerodible or biodegradable material, known in the art, such as a synthetic polymer, e.g., a polyester (such as PLGA), a poly(anhydride), or a polycaprolactone.

In other embodiments, the reservoir cap comprises an electrically conductive material, and the device includes means for actively disintegrating the reservoir cap. As used herein, the term “disintegrate” is used broadly to include without limitation degrading, dissolving, rupturing, fracturing or some other form of mechanical failure, as well as fracture and/or loss of structural integrity of the reservoir cap due to a chemical reaction or phase change (e.g., melting or vaporization), unless a specific one of these mechanisms is indicated. Electrothermal ablation is a preferred form of active disintegration, the means of which are taught in U.S. patent application Publication No. 2004/0121486 A1 to Uhland et al., which is incorporated herein by reference in its entirety. In another embodiment, the disintegration comprises corrosion, e.g., electrochemically induced oxidation and dissolution. Examples of suitable reservoir cap opening technologies and the activation means therefor are further described in U.S. Pat. No. 5,797,898, U.S. Pat. No. 6,527,762, and U.S. Pat. No. 6,491,666, U.S. patent application Publication Nos. 2004/0121486, 2002/0107470 A1, 2002/0072784 A1, 2002/0138067 A1, 2002/0151776 A1, 2002/0099359 A1, 2002/0187260 A1, and 2003/0010808 A1; PCT WO 2004/022033 A2; PCT WO 2004/026281; and U.S. Pat. Nos. 5,797,898; 6,123,861; and 6,527,762, all of which are incorporated by reference herein.

In a preferred embodiment, a discrete reservoir cap completely covers/plugs a single reservoir opening. In another embodiment, a discrete reservoir cap covers two or more, but less than all, openings in a single reservoir, for instance where a single reservoir has multiple, adjacent openings, in the same surface, in a single reservoir.

In devices where release is passively controlled, the reservoir caps are formed from a material or mixture of materials that degrade, dissolve, or disintegrate over time, or that do not degrade dissolve, or disintegrate, but are permeable or become permeable to the therapeutic or prophylactic agent. Representative examples of reservoir cap materials include polymeric materials, and non-polymeric materials such as porous forms of metals (e.g., trabecular metal, a porous tanatalum), semiconductors, and ceramics. Passive semiconductor barrier layer materials include nanoporous or microporous silicon membranes.

In devices where release is actively controlled, the reservoir cap includes any material that can be disintegrated or permeabilized in response to an applied stimulus (e.g., electric field or current, magnetic field, change in pH, or by thermal, chemical, electrochemical, or mechanical means). Examples of suitable reservoir cap materials include gold, titanium, platinum, tin, silver, copper, zinc, alloys, and eutectic materials such as gold-silicon and gold-tin eutectics. Any combination of passive or active barrier layers can be present in a single device.

In one active release embodiment, the reservoir caps are in the form of a thin metal film. In another, the reservoir caps are made of multiple metal layers, such as a multi-layer/laminate structure of platinum/titanium/platinum. For example, the top and bottom layers could be selected for adhesion layers on (typically only over a portion of) the reservoir caps to ensure that the reservoir caps adhere to/bonds with both the substrate area around the reservoir openings, reservoir cap supports, and a dielectric overlayer. In one specific example, the structure is titanium/platinum/titanium/platinum/titanium, where the top and bottom layers serve as adhesion layers, and the platinum layers provide extra stability/biostability and protection to the main, central titanium layer. The thickness of these layers could be, for example, about 300 nm for the central titanium layer, about 40 nm for each of the platinum layers, and between about 10 and 15 nm for the adhesion titanium layers.

Publications cited herein are incorporated by reference. Modifications and variations of the methods and devices described herein will be obvious to those skilled in the art from the foregoing detailed description. Such modifications and variations are intended to come within the scope of the appended claims.

We claim:

1. An implantable prosthetic device for controlled drug delivery comprising:
a prosthetic device body having at least one outer surface area;
two or more discrete reservoirs located in spaced apart positions across at least a portion of the outer surface area, the reservoirs formed with an opening at the surface of the device body and extending into the device body; and
a release system disposed in the reservoirs which comprises at least one therapeutic or prophylactic agent,
wherein following implantation into a patient the therapeutic or prophylactic agent is released in a controlled manner from the reservoirs.

2. The device of claim 1, wherein the prosthetic device body is a joint prosthesis or part thereof.

3. The device of claim 2, wherein the prosthetic device body comprises a hip prosthesis or part thereof.

4. The device of claim 2, wherein the prosthetic device body comprises a knee prosthesis or part thereof.

5. The device of claim 1, wherein the prosthetic device body comprises a vertebral or spinal disc prosthesis, spinal cage, or part thereof.

6. The device of claim 1, wherein the prosthetic device body comprises a surgical staple, screw, or plate.

7. The device of claim 1, wherein the prosthetic device body and surface area in which the reservoirs are defined comprises a biocompatible material selected from metals, polymers, ceramics, and combinations thereof.

8. The device of claim 7, wherein the device body comprises a stainless steel, a chrome-cobalt alloy, a titanium alloy, a ceramic, or an ultra high molecular weight polyethylene.

9. The device of claim 1, wherein the prosthetic device body comprises a porous surface region.

10. The device of claim 9, wherein the prosthetic device body further comprises a non-porous region, at least part of which is located beneath the porous region.

11. The device of claim 1, wherein the therapeutic or prophylactic agent comprises an antibiotic agent.

12. The device of claim 1, wherein the therapeutic or prophylactic agent comprises one or more growth factors.

13. The device of claim 1, wherein the therapeutic or prophylactic agent is a self-propagating agent.

14. The device of claim 1, wherein the release of the therapeutic or prophylactic agent is passively controlled.

15. The device of claim 1, wherein the release of the therapeutic or prophylactic agent is actively controlled.

16. The device of claim 1, wherein the release system further comprises one or more matrix materials.

17. The device of claim 16, wherein the matrix material comprises one or more synthetic polymers.

18. The device of claim 16, wherein the one or more matrix materials comprises a biodegradable, bioerodible, water-soluble, or water-swellable matrix material.

19. The device of claim 16, wherein the therapeutic or prophylactic agent is distributed in the matrix material and the matrix material degrades or dissolves in vivo to controllably release the therapeutic or prophylactic agent.

20. The device of claim 16, wherein the release system is provided in two or more layers having different compositions.

21. The device of claim 20, wherein each of the at least two reservoirs comprises at least two layers which comprise the one or more therapeutic or prophylactic agents and at least one layer of a degradable or dissolvable matrix material which does not comprise the one or more therapeutic or prophylactic agents.

22. The device of claim 20, wherein at least a first therapeutic or prophylactic agent is contained in a first layer of the two or more layers, and wherein a second therapeutic or prophylactic agent is contained in a second layer of the two or more layers.

23. The device of claim 1, wherein the therapeutic or prophylactic agent is heterogeneously distributed in the reservoir.

24. The device of claim 1, wherein the therapeutic or prophylactic agent is homogeneously distributed in the reservoir.

25. The device of claim 1, wherein the quantity of therapeutic or prophylactic agent provided for release from at least a first of the reservoirs is different from the quantity of the therapeutic or prophylactic agent provided for release from at least a second of the reservoirs.

26. The device of claim 1, wherein the kinetics of release of one of the therapeutic or prophylactic agents from at least a first of the reservoirs is different from the kinetics of release of the therapeutic or prophylactic agent from at least a second of the reservoirs.

27. The device of claim 1, wherein a first therapeutic or prophylactic agent is in at least one of the reservoirs and a second therapeutic or prophylactic agent is in at least one other of the reservoirs, the first therapeutic or prophylactic agent and the second therapeutic or prophylactic agent being different in kind or dose.

28. The device of claim 1, further comprising one or more discrete reservoir caps positioned over or disposed in the reservoir openings, wherein the time and/or rate of release of the therapeutic or prophylactic agent is controlled by the reservoir caps.

29. The device of claim 28, wherein the reservoir caps are non-porous.

30. The device of claim 29, wherein the reservoir caps comprise a biodegradable or bioerodible polymer.

31. The device of claim 30, wherein the biodegradable or bioerodible polymer is selected from the group consisting of poly(lactic acids), poly(glycolic acids), poly(lactic-co-glycolic acids), poly(caprolactones), poly(anhydrides), and mixtures thereof.

32. The device of claim 28, wherein the reservoir caps have a thickness between 0.1 and 100 microns.

33. The device of claim 29, wherein at least one discrete reservoir cap is formed of a first material and at least one other discrete reservoir cap is formed of a second material, wherein the first material has a different degradation or dissolution rate compared to the second material.

34. The device of claim 29, wherein at least one discrete reservoir cap has a first thickness and at least one other discrete reservoir cap has a second thickness, wherein the first thickness is different from the second thickness, thereby providing different times of release of the one or more therapeutic or prophylactic agent from the reservoirs covered respectively by the discrete reservoir cap having the first thickness and the discrete reservoir cap having the second thickness.

35. The device of claim 1, comprising at least two rows of at least two reservoirs.

36. The device of claim 35, wherein a first release system is in each of the at least two reservoirs of a first row and a second release system is in each of the at least two reservoirs of the other of the at least two rows other of the reservoirs, the first release system releasing the one or more therapeutic or prophylactic agents at a rate or in a dosage amount different from release of the one or more therapeutic or prophylactic agents from the second release system.

37. The device of claim 1, wherein the reservoirs are micro-reservoirs.

38. The device of claim 29, wherein the reservoir caps comprise a metal film.

39. The device of claim 38, further comprising control means to actively disintegrate the reservoir cap.

40. The device of claim 1, wherein the outer surface comprises a porous region, and the release system comprises a calcium phosphate compound, a bone morphogenic protein or a recombinant version thereof, or a combination thereof.

42. The device of claim 40, wherein the reservoirs comprise macroreservoirs.

43. The device of claim 42, wherein the pores of the porous surface region comprise a bone morphogenic protein or a recombinant version thereof.

44. A method of releasing a therapeutic or prophylactic agent from a prosthetic device in vivo comprising:
implanting the prosthetic device of claim 1 at a site in a patient in need thereof; and
releasing the therapeutic or prophylactic agent from the prosthetic device.

45. An implantable prosthetic device for controlled drug delivery comprising:
a prosthetic device body having at least one outer surface area;
two or more discrete microreservoirs located in spaced apart positions across at least a portion of the outer surface area, the microreservoirs formed with an opening at the surface of the device body and extending into the device body;
a release system disposed in the microreservoirs which comprises at least one therapeutic or prophylactic agent; and
a plurality of discrete, non-porous reservoir caps located over the release system in the microreservoirs,
wherein release of the therapeutic or prophylactic agent following implantation of the device into a patient is controlled by the reservoir caps.

46. The device of claim 45, wherein the release system further comprises a matrix material, which further controls release of the therapeutic or prophylactic agent in vivo.

47. An implantable prosthetic device for controlled drug delivery comprising:
a prosthetic device body having at least one outer surface area;
two or more discrete microreservoirs located in spaced apart positions across at least a portion of the outer surface area, the microreservoirs formed with an opening at the surface of the device body and extending into the device body; and
a release system disposed in the reservoirs which comprises at least one therapeutic or prophylactic agent and a biodegradable or bioerodible matrix material;
wherein release of the therapeutic or prophylactic agent in vivo following implantation of the device into a patient is controlled by the matrix material.

48. The device of claim 47, further comprising a plurality of discrete, non-porous reservoir caps located over the release system in the reservoir reservoirs, wherein the reservoir caps further control release of the therapeutic or prophylactic agent in vivo.

49. The device of claim 47, wherein the release system is provided in two or more layers having different compositions.

50. The device of claim 47, wherein at least a portion of the device body is non-porous.

51. The device of claim 47, wherein at least a portion of the device body comprises a metal.

52. The device of claim 47, wherein the device body comprises at least one porous surface region.

53. A method for in vivo local delivery of a therapeutic or prophylactic agent in the treatment of orthopedic tissues comprising:
implanting at a orthopedic tissue site a tip portion of a tube which comprises a first end and a distal second end, wherein the tip portion has located therein a plurality of discrete reservoirs containing a therapeutic or prophylactic agent, the reservoirs having openings sealed by a plurality of discrete reservoir caps; and
actively and selectively disintegrating the reservoir caps to initiate release of the therapeutic or prophylactic agent at the tissue site.

54. The method of claim 53, wherein the orthopedic tissue site is a joint or spinal disc.

55. The device of claim 1, wherein the prosthetic device body is a dental implant.

56. The device of claim 55, wherein the release system comprises a bone morphogenic protein, a growth factor, an anti-infective agent, or a combination thereof.
